(19) United States
(12) Patent Application Publication
(10) Pub. No.: US 2020/0269021 A1
(43) Pub. Date: Aug. 27, 2020

(54) ESOPHAGUS FOOD DISIMPACTOR

(71) Applicant: Paul Hagan, JR., Holly Springs, NC (US)

(72) Inventor: Paul Hagan, JR., Holly Springs, NC (US)

(21) Appl. No.: 16/282,269

(22) Filed: Feb. 21, 2019

(51) Int. Cl. A61M 25/10

(52) U.S. Cl. CPC A61M 25/1002; A61M 25/0012; A61M 25/104

(57) ABSTRACT

An esophageal catheter for dislodging food from a user is presented. The esophageal catheter has an elongated body that has a distal end and a proximal end. The esophageal catheter also has an inflation member, the inflation member has a distal end and a proximal end and is attached to the elongated body. Further, the esophageal catheter has a guide member positioned at the distal end of the elongated body. Also, the esophageal catheter has a cone-like member having a plurality of arms and positioned at the proximal end of the inflation member. When the cone-like member is in an expanded state, the cone-like member extends from one side of an esophagus of the user to a second side of the esophagus of the user. The esophageal catheter is an adjunctive instrument and technique and not as a replacement for other instruments or techniques to remove esophageal boluses.

### FIELD OF THE INVENTION

This invention relates to esophageal catheters. More particularly, it relates to esophageal catheters for dislodging food from an esophagus.

### BACKGROUND

Balloon dilatation catheters are used for a wide variety of medical procedures, typically to enlarge a body lumen that has become obstructed. For example, balloon dilatation catheters commonly are used in angioplasty procedures to enlarge the lumen of a blood vessel constricted or stenosed by arteriosclerosis. Recent years have seen significant advances in percutaneous transluminal coronary angioplasty in which a dilatation catheter having a relatively inelastic polymeric balloon on its distal end is advanced percutaneously into the patient's arterial system and into the coronary artery to be treated. The balloon is inserted into the stenosis and is inflated to affect the dilatation. Balloon dilatation catheters also are used to dilate other body lumens, such as, for example, in the esophageal tract, blood vessels and the like.

Balloon dilatation catheters often are used in conjunction with another tubular guide member through which the dilatation catheter is inserted and guided to the desired location in the body lumen of the patient. Dilatation balloons used to treat a patient's gastrointestinal tract often are inserted into the patient through an endoscope. Endoscopes vary in length, depending on their intended purpose. For example, a typical multi-purpose gastrointestinal endoscope may be of the order of four and one-half to five feet long and may be of the order of one-inch diameter.

It is formed from a plurality of articulated sections so that it is flexible. Controls, such as pull wires, are provided at the proximal end of the endoscope to control the shape of the distal end to steer and directionally control the endoscope. The endoscope typically has multiple channels for various functions and usually includes optical fiber channels to illuminate and permit visual observation of the patient's gastrointestinal tract. One or more channels, often only a few millimeters in diameter, also are provided through which instruments may be inserted into the patient such as, for example, miniature biopsy forceps, snares, needles, brushes, balloon dilatation catheters and others.

An illustrative procedure may involve dilatation of the esophagus in order to enlarge a constriction caused, for example, by a tumor, esophagitis or other condition. In the procedure, the endoscope is passed through the patient's throat and into the esophagus. The progress of the endoscope is monitored visually through the fiber optic and illumination system.

When the obstructed portion of the esophagus is reached, a dilatation catheter having a balloon at its distal end is passed through the instrument channel, with the balloon deflated, so that the balloon exits the distal end of the endoscope and passes through the obstruction. Once the balloon is placed within the obstruction, it is inflated to dilate the obstructed portion of the esophagus. The balloon must be deflated to a low profile in order for it to be passed through the instrument channel. Typically, the balloon is deflated by applying negative pressure to the balloon through an inflation/deflation lumen that extends from the proximal end of the catheter to the interior of the balloon.

Among the difficulties with dilatation balloons is the configuration that is assumed by the balloon when it is deflated. Typically, the balloon forms a pair of opposed, radially extending flat wings when it collapses under the influence of negative pressure. In order to insert the balloon into the instrument channel (or guide catheter lumen), the physician typically will manually wrap the wings about the catheter shaft and will insert the balloon catheter into the channel or guide catheter lumen in that configuration. The reduced profile also facilitates insertion of the balloon end of the catheter through the constriction.

After the balloon has been inflated to perform the dilatation, it typically is deflated and, when that is done, it tends to assume the two-wing configuration. The wings typically extend diametral a distance substantially larger than the diameter of the channel. If it is desired to leave the endoscope in place and withdraw the dilatation catheter through the channel, that may present problems if the wings fail to wrap about the catheter shaft as the balloon is drawn into the channel. The wings may become caught at the distal end of the channel or otherwise interfere with smooth withdrawal. Withdrawal of the catheter may be necessary when it is desired to change catheters during the procedure, for example, as when the physician decides that further dilatation with a larger diameter balloon is required.

### BRIEF DESCRIPTION OF THE DRAWINGS

FIG. 1 is an exemplary esophageal catheter.

FIG. 2 is an exemplary esophageal catheter with an inflation member being inflated.

FIG. 3 is an exemplary esophageal catheter with a fully inflated inflation member.

FIG. 4 is an esophageal tube with impacted food.

FIG. 5 is an esophageal tube with a esophageal catheter being placed to remove the impaction.

FIG. 6 is an esophageal tube with an esophageal catheter having its inflation member inflated.

FIG. 7 is the removal of the esophageal catheter from the esophageal tube.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

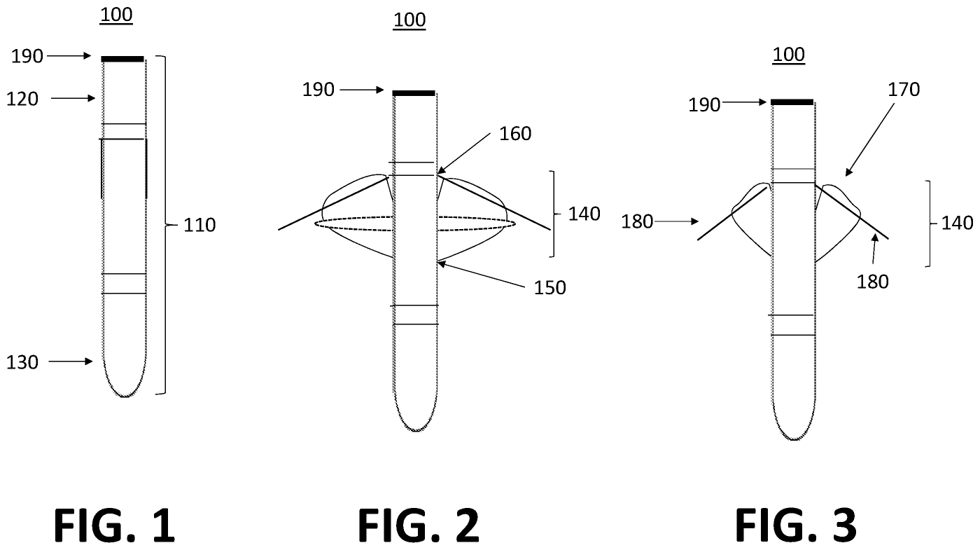

Referring to FIG. 1, an exemplary esophageal catheter 100 is shown. The esophageal catheter 100 is useful for various actions but in particular to this application the esophageal catheter 100 is useful when a person has food lodged in their esophagus, it can be used to remove the impaction of the food and open the esophagus.

The esophageal catheter 100 can be made of any material such as surgical plastic and the like. Further the esophageal catheter 100 can be bendable such that it is able to easily maneuver into and around the esophagus but still stiff enough to be able to dislodge the food in the esophagus to open the esophagus up and provide relief to the person.

The esophageal catheter 100 has a body 110, the body may be of any shape and size but is preferably elongated and slender. Thus, the esophageal catheter 100 is more easily placed and maneuvered when inside of the esophagus.

The elongated body 110 of the esophageal catheter 100 has a proximal end 120 and a distal end 130. The proximal end 120 of the esophageal catheter 100 in open and has a rubber gasket 190. The rubber gasket 190 is useful a guidance near the proximal end 120 of the esophageal catheter 100 to allow for safe guidance of the esophageal catheter 100 into the stomach without perforation and easily positional. The elongated body 110 of the esophageal catheter 100 is the NOVEL Functioning or business end. The distal end 130 may further comprise a rubber guidance tip that allows the catheter 100 to slide past food while permitting placement of the inflation member 140 (see FIG. 2).

Moving now to FIG. 2, an exemplary esophageal catheter 100 is shown. The esophageal catheter 100 is useful for various actions but in particular to this application the esophageal catheter 100 is useful when a person has food lodged in their esophagus, it can be used to remove the impaction of the food and open the esophagus.

The esophageal catheter 100 can be made of any material such as surgical plastic and the like. Further the esophageal catheter 100 can be bendable such that it is able to easily maneuver into and around the esophagus but still stiff enough to be able to dislodge the food in the esophagus to open the esophagus up and provide relief to the person.

The esophageal catheter 100 has an inflation member 140. The inflation member 140 is useful to push or compress the food lodged in the esophagus to the sides of the esophagus and allow the food to be dislodged and be pushed to the stomach. The inflation member 140 is also useful as it can be deflated and the esophageal catheter 100 to be easily removable.

The inflation member 140 has a proximal end 160 and a distal end 150. The inflation member 140 may be of any shape but is preferably conical. The inflation member 140 may measure approximately 4-5 cm in magnitude. The inflation member 140 is located substantially near the middle of the elongated body 110 but may be slightly closer to the proximal end 120 of the elongated body 110. The esophageal catheter 100 further has a rubber gasket 190. The rubber gasket 190 is useful a guidance near the proximal end 120 of the esophageal catheter 100 to allow for safe guidance of the esophageal catheter 100 into the stomach without perforation and easily positional. The rubber gasket 190 and distal end 130 may allow for safe guidance of the esophageal catheter 100 into the stomach without perforation while remaining easily positional.

Now moving to FIG. 3, a further showing of the esophageal catheter 100 is presented. The esophageal catheter 100 is useful for various actions but in particular to this application the esophageal catheter 100 is useful when a person has food lodged in their esophagus, it can be used to remove the impaction of the food and open the esophagus.

The esophageal catheter 100 can be made of any material such as surgical plastic and the like. Further the esophageal catheter 100 can be bendable such that it is able to easily maneuver into and around the esophagus but still stiff enough to be able to dislodge the food in the esophagus to open the esophagus up and provide relief to the person.

The esophageal catheter 100 has a rubber gasket 190. The rubber gasket 190 may resemble or act as a guidance portion. The rubber gasket 190 may measure approximately 5 cm. The rubber gasket 190 is useful a guidance near the proximal end 120 of the esophageal catheter 100 to allow for safe guidance of the esophageal catheter 100 into the stomach without perforation and easily positional.

The esophageal catheter 100 has an inflation member 140. The inflation member 140 is located substantially near the middle of the elongated body 110 but may be slightly closer to the proximal end 120 of the elongated body 110.

The inflation member 140 has a cone-like wing 170. The inflation member 140 further has a plurality of arms 180. The inflation member 140 along with the plurality of arms 180 and cone-like wing 170 may be responsible for pressing the food bolus against the esophagus wall, thereby dilating the esophagus and pushing food from the esophagus into the stomach.

The cone-like wing 170 of the inflation member 140 is near the proximal end 160 of the inflation member 140. The cone-like wing 170 will push the food, when inflated, into the stomach, much like when using a plunger to unstop a drain.

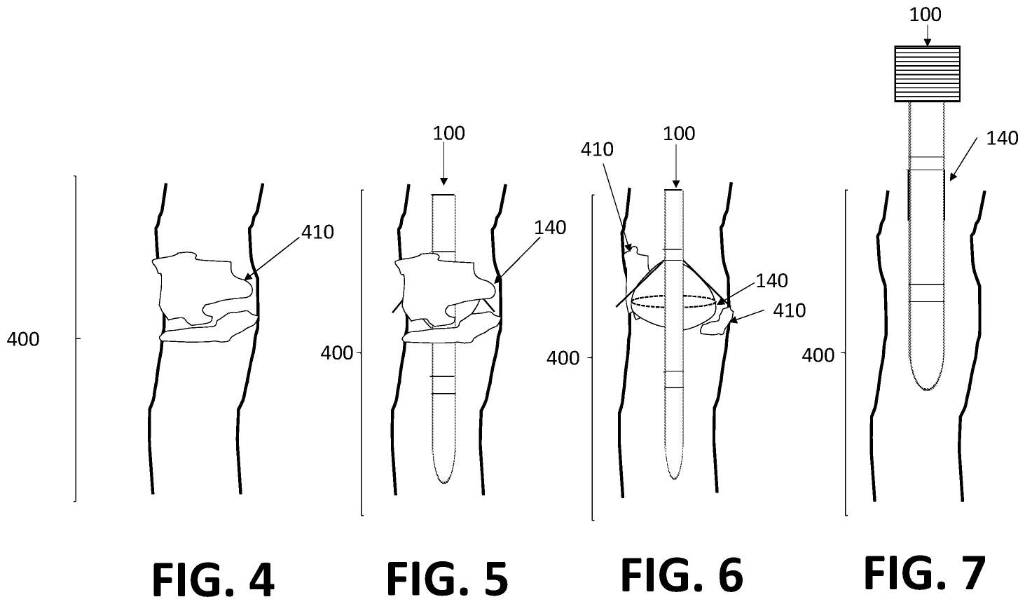

FIG. 4, FIG. 5, FIG. 6 and FIG. 7 show the use of the esophageal catheter 100 when the esophagus 400 has food 410 lodged in it.

FIG. 4 shows a person has just eaten or taken in food 410. The food 410 has become lodged or stuck in the esophagus 400. The person cannot swallow, thereby possibly panicking and at worse may be losing air while saliva and fluid accumulate in the esophagus.

In FIG. 5, a GI doctor, or other person versed or skilled in the use of the esophageal catheter 100 comes to the person with the food 410 lodged in their esophagus 400. The esophageal catheter 100 is inserted through the mouth of the person and is maneuvered into the esophagus 400.

Now in FIG. 6, the doctor or other person inflates the cone-like wing 170 of the inflation member 140. The cone-like wing 170 is expanded and reaches the sides of the esophagus 400. The food 410 is first pushed to the side and then once fully inflated, the cone-like wing 170 of the inflation member 140 pushes the food down towards the stomach. At this point, the food 410 is displaced and either resides near the sides of the esophagus 400 or is pushed into the stomach. Relief may be achieved by a back and forth motion of the exemplary catheter 100. The person is now relieved of the pressure and discomfort of the food 410 lodged in their esophagus 400. Breathing now resume comfortably and the person relaxes.

In FIG. 7, the inflation member 140 is deflated. The cone-like wing 170 is pulled back to the sides of the elongated body 110 of the esophageal catheter 100, thus the slender aspects of the esophageal catheter 100 is returned into the endoscope and allows for the doctor or other person to remove the esophageal catheter 100 from the esophagus 400 of the person that had the food 410 lodged in their esophagus 400. This was a safe, quick and painless procedure that is easily mobile to whatever location that one goes.

The numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An esophageal catheter for disimpacting food from a user, comprising:
an elongate body having a distal end and a proximal end;
an inflation member, said inflation member having a distal end and a proximal end and attached to said elongate body;
a guide member, said guide member positioned at said distal end of said elongate body;
a cone-like member, said cone-like member having a plurality of arms and positioned at said proximal end of said inflation member; and
wherein when in an expanded state, said cone-like member extends from one side of an esophagus of said user to a second side of said esophagus of said user.

2. The esophageal catheter of claim 1, wherein said inflation member is a balloon.

3. The esophageal catheter of claim 1, wherein said plurality of arms comprises 2 arms.

4. The esophageal catheter of claim 1, wherein said guide member is made of a soft material.

5. The esophageal catheter of claim 3, wherein said guide member is stiff.

6. The esophageal catheter of claim 4, wherein said guide member is made of a rubber material.

7. The esophageal catheter of claim 1, wherein said cone-like member is configured to push an amount of food out of said esophagus of said user.

8. The esophageal catheter of claim 1, wherein said distal end of said elongate body is tapered.

9. The esophageal catheter of claim 1, wherein said proximal end of said elongate body is non-tapered.

10. A method for disimpacting food from an esophagus, said method comprising the steps of:
inserting an endoscope into a patient's mouth;
advancing said endoscope to a bolus of food in said esophagus;
inserting an esophageal catheter through said endoscope;
advancing said esophageal catheter beyond said bolus so that a deflated balloon portion of said esophageal catheter is at a position in said esophagus that is next to said bolus; and
inflating said deflated balloon portion so that said bolus is impacted to a side wall of said esophagus.

11. The method of claim 9, further comprising the steps of:
expanding said cone-like member; and
moving said esophageal catheter in a back and forth motion so as to push said bolus of food out of said esophagus.
